(19) United States
(12) Patent Application Publication
(10) Pub. No.: US 2022/0312711 A1
(43) Pub. Date: Oct. 6, 2022

(54) TRACEABLE COMPOSITE FOR MARKING SEEDS AND PLANTS

(71) Applicant: SECURITY MATTERS LTD., D.N. Revel Eilot (IL)

(72) Inventors: Yair GROF, Rehovot (IL); Nataly TAL, Nes Ziona (IL); Mor KAPLINSKY, Herzliya (IL); Ron DAFNI, Tel Aviv (IL); Yifat BAREKET, Reut (IL); Michal FIRSTENBERG, Rehovot (IL); Tehila NAHUM, Holon (IL); Hagit SADE, Ramat Gan (IL); Nadav YORAN, Tel Aviv (IL); Haggai ALON, Kibbutz Naan (IL)

(21) Appl. No.: 17/639,397

(22) PCT Filed: Sep. 2, 2020
(86) PCT No.: PCT/IL2020/050956

§ 371 Date: Mar. 1, 2022

Related U.S. Application Data
(60) Provisional application No. 62/894,892, filed on Sep. 2, 2019.

(51) Int. Cl. A01H 4/00

(52) U.S. Cl. CPC A01H 4/006

(57) ABSTRACT

The invention concerns compositions and methods for authenticating an agricultural product.

### TECHNOLOGICAL FIELD

The present invention is in the field of authentication of seeds, plants and other agricultural products.

### GENERAL DESCRIPTION

International trade of seeds and plant-derived products is one of the major forces in the global economy and the demand is increasing in both developed and developing countries. The massive demand for viable and stable seeds that can germinate in high percentages to produce health plants producing commercial products as flowers, fruits and vegetables has resulted in the development of unique methods of production and use.

One of the main problems associated with the standardization of seeds and other plant materials resides in black market products that cannot be simply and cost effectively differentiated from. To achieve efficient standardization and proper differentiation between agricultural products such as seeds of low quality and seeds produced to yield products of commercial usefulness, the inventors of the technology disclosed herein have developed a method of authenticating viable agricultural products, such as seeds and other explant materials, for the purpose of monitoring on-sale marketing of viable seeds and explants and for identifying illicitly produced seeds or explants.

Methods of the invention generally provide means for associating at least one X-Ray Fluorescence (XRF) marker to such seeds and explants, wherein the association does not only provide the means for directly authenticating the seed or explant, but also enables identification of plants or plant materials derived from the seed or explant. The detection of the marker in the seed or in a seedling derived therefrom or in a plant or in an isolate (extract) derived from the seed or plant, may be for a purpose such as authentication, brand protection, proving the origin of the plant as well as for managing supply chain of plants and plant ingredients and products.

Thus, in a first aspect the invention provides an agricultural composition comprising at least one XRF identifiable marker (referred to interchangeably herein as a marker). The composition, as explained herein, may be adapted for application in a variety of forms. For marking living plants with an XRF identifiable marker, the invention provides several alternative methods, as follows:
Marking a seed or a seedling under conditions permitting development of a plant enriched with the XRF identifiable marker;
Enriching a living plant with the XRF identifiable marker through irrigation or fertilization; and
Applying a marker composition to a surface of a living plant, pre- or post-harvest, e.g., by spraying.

The invention further provides a composition comprising at least one XRF identifiable marker, as defined herein, wherein the composition is configured for application onto an explant or a living plant surface.

Also contemplated is a composition adapted to be taken up by a living plant, the composition comprising at least one XRF identifiable marker, as defined herein.

Compositions of the invention, used in accordance with methods of the invention, are generally adapted for agricultural use. As such, the agricultural composition may comprise an amount of the XRF identifiable marker along with at least one agriculturally acceptable carrier that is capable of securing or permitting or allowing association between, e.g., an explant or a seed and the marker, or a carrier that solubilizes or contains the marker in its presented form. The carrier and/or the marker may be selected to be water soluble or water insoluble. In cases where the marker is water soluble, it may decompose in the presence of water and be taken up by the developing germ. Also, the carrier may be selected amongst materials known as nutrients or fertilizers that can assist in the uptake of the marker by a developing seedling.

In some embodiments, an agricultural composition of the invention may thus comprise at least one XRF identifiable marker, at least one carrier and optionally at least one additive. The at least one additive may be selected from adhesive materials, coloring materials, pesticides, fertilizers, nutrients and other agricultural additives as may be known in the art.

The XRF marker present in an agricultural composition of the invention may be any marker material that include atoms which are detectable by X-Ray Fluorescence (XRF) spectroscopy, wherein an X-ray or Gamma-ray radiation is directed towards a sample, e.g., of the seeds or explant, and a response X-ray signal therefrom is detected and analyzed. An XRF spectrometer (analyzer) may detect the presence of and measure the concentration of the markers on the surface of the seed or explant, or generally on the surface of any part of the plant with which the marker is associated.

As the presence of the at least one XRF identifiable marker is to be distinguished from materials that naturally exist in the seed or the explant to be authenticated, the XRF identifiable marker is either selected amongst such materials that are not typically found in the seed or explant, nor in an environment in which the seed or explant is grown (including soil and water), nor in a fertilizer or a nutrient that are used in the process of managing their growth, or is provided in a composition of the invention in an amount or concentration that is greater than the amount present in the plant or the environment, such that when the marker is taken up by the seed or explant, the signal obtained upon reading the XRF signal received from the marker is distinguishable from a signal received from, e.g., a naturally occurring concentration.

In some embodiments, the at least one XRF identifiable marker is selected not to be a component of a plant nutrient (any chemical element or compound necessary for plant growth or plant metabolism; such as macronutrients and micronutrients), a material naturally present in the living plant or in a soil in which the living plant is grown or is not a material used in management of growth (such as a fertilizer, a nutrient, etc).

Additionally, the at least one XRF identifiable marker may be a combination of two or more such markers provided at a ratio that distinguishes one or more of the markers from any one or more markers that may be present naturally in the seed, explant or environment of growth, as detailed herein.

In some embodiments, the at least one XRF identifiable marker may be selected amongst metal carbides, metal cyanides, metal sulfides, metal sulfites, metal sulfates, metal hydrogen sulfate, metal acetates, metal carbonates, metal bicarbonates, metal oxide, bi- or tri-metal atom molecules, metalorganics, metal halides, metal nitrides, metal nitrites, metal nitrates, metal silicides, metal hydroxides, metal peroxides, metal permanganates, metal tellurides, metal carbonyls, metal silicates, metal phosphates, metal dihydrogen phosphates, metal borides, metal oxalate, metal dichromate and metal chromate.

In some embodiments, the at least one XRF identifiable marker is at least one atom selected from groups of atoms designated coinage, triels, prictogen, chalcogen, or tetrels.

In some embodiments, the at least one XRF identifiable marker is selected from hypochlorites, chlorite salts, chlorate salts, perchlorate salts, bromide salts and bromate salts.

In some embodiments, the at least one XRF identifiable marker is a salt form of at least one metal having an atomic weight larger then Mg. In some embodiments, the metal is selected from Bi, Ge, Ga, St, Mo, Y, Nb, Cr, Zn, As, Sr, Se, Zr, Sn, Sb and Hf.

In some embodiments, the at least one XRF identifiable marker is an oxide form of at least one metal selected from Bi, Ge, Ga, St, Mo, Y and Nb.

In some embodiments, the at least one XRF identifiable marker is one or more of bismuth germanium oxide (Bi₄Ge₃O₁₂), gallium oxide (Ga₂O₃), strontium molybdate (SrMoO₄), yttrium oxide (Y₂O₃), niobium oxide (Nb₂O₅), bismuth (III) citrate (BiC₆H₅O₇) and bismuth oxide (Bi₂O₃).

In some embodiments, the at least one XRF identifiable marker is a bromide-containing molecule, which may be a bromide salt or a bromo-substituted organic or inorganic molecule.

In a composition of the invention, the number of different markers used and the relative quantity of each such marker may vary depending, inter alia, on the particular intended use, the seed or explant to be authenticated, the conditions of seed growth and the time period extending between, e.g., the marking of a seed and time of germination. In some embodiments, a composition of the invention may comprise a single XRF identifiable marker. In other embodiments, the number of XRF identifiable markers may be two or more, wherein each of the two or more markers may be different in amount and constitution (e.g., metal ion, counter ion and others).

Compositions of the invention may be used for a variety of agricultural applications. A composition comprising the at least one XRF identifiable marker may be configured for application onto a surface of a viable plant (i.e., a living plant or a seedling), a seed or an explant to enable authentication thereof.

As used herein, an explant is any part of any plant that can be developed into a viable plant. The explant may be selected from shoot tips, axillary buds, somatic embryos, seeds and others. In some embodiments, the explant is a seed.

The “plant” of any aspect or embodiment disclosed herein, in reference to a living plant or to a seed or an explant to be developed into a plant, is a member of the plant kingdom, mainly selected from vascular plants. The plant may be a flowering plant, a vegetable producing plant, a fruit producing plant, a medicinal plant, a horticultural plant, and others. The plant may be a deciduous plant, semi-deciduous or an evergreen.

In some embodiments, the plant is a flowering plant, a vegetable producing plant, or a fruit producing plant.

In some embodiments, the plant is selected from cannabis, aloe vera, jojoba, citronella, Echinacea, rose, neem, Tabaco, cananga, candamone, primrose, cinnamon, eucalyptus, cherry, lavender, vanilla, lilach, ginger, nutmeg, spearmint, peppermint, melon, watermelon, mint, and others.

In some embodiments, the plant is cannabis.

In some embodiments, the seed or explant is of a deciduous plant or an evergreen. In some embodiments, the seed or explant is of cannabis.

A composition of the invention may also be adapted to enable uptake of the at least one XRF identifiable marker and/or the carrier by a living plant, namely to enable the marker to be absorbed by a seed during the germination process, or be absorbed by a seedling during its development, or be absorbed by a plant in any of its growing cycles in a way that enables movement of the marker from the seed coating or the environment in which the seed or plant is grown into the plant.

The invention further provides a method for XRF marking of an explant, as defined, the method comprising forming a coating of at least one XRF identifiable marker, wherein the at least one XRF identifiable marker is optionally comprised within at least one carrier material.

In some embodiments, the explant is a seed.

The invention thus further provides a seed coated on its surface with a coating comprising at least one XRF identifiable marker.

Also provided is a seed comprising an absorbed amount of at least one XRF identifiable marker, as defined herein.

A coating comprising the at least one XRF identifiable marker may be formed around the surface of the seeds, or explants, by any means known in the art. While the coating may consist the at least one XRF identifiable marker, in some embodiments, the at least one XRF identifiable marker is comprised within, embedded or dispersed in at least one carrier material that acts as a matrix embedding the marker. The carrier may further comprise at least one additive, as disclosed herein.

The at least one carrier material may be any material used in seed treatment to protect the seed from pests and diseases. The at least one carrier may be selected amongst natural and synthetic materials. In some embodiments, the at least one carrier material is selected amongst natural or synthetic polymers. Non-limiting examples of such materials may include cellulose and cellulose-derived materials, chitosan, acacia gum, starch, polyethylene glycol, polyvinyl acetate, polyvinylpyrrolidone, and others.

Typically, the coating formed on the surface of the seeds or explant comprises the at least one carrier and an effective or sufficient amount of the at least one XRF identifiable marker. The amount of the marker is selected to permit effective reading by an XRF spectrometer, as detailed hereinbelow.

In some embodiments, the coating is formed prior to or during germination.

The concentration of each of the marker materials can be measured by an XRF spectrometer (analyzer), therefore a marking composition including a plurality of markers wherein each can be present in a range of concentrations can be used to encode information relating for example, to the seed or explant, its batch, its origin, date of seeding and/or harvesting, its intended use, its destination or various processing facilities and others. The marking may also be used to indicate the particular strain of a plant or germ facilitating in situ identification of the plant (for example, in the field or growing facility).

In some embodiments, the concentration of the XRF identifiable marker is between 0.0001 and 10 wt %, relative to the total weight of the coating composition. The thickness of the coating layer of the seed may range between tens of microns and few millimeters.

The coating may be formed on any one region of the seed or the explant. The region of the seed or the explant onto which the coating is applied may be selected to provide a patterning parameter that distinguishes the marked seed or explant from unmarked or forged seeds or explants. In some embodiments, a coating is formed over the full surface of the seed or explant. In some embodiments, a coating is formed on one or multiple spaces apart regions of the seed or explant.

The coating may be formed by any means known in the art, including by spraying, brushing, soaking, dipping in a vessel or a container, topdressing, or incorporation in a sand surrounding the seeds by plow or rototiller.

Were seeds are concerned, for example, they may by coated by a composition comprising the marker(s), which can be applied to the surface of the seeds by spraying, brushing, soaking or dipping in a vessel or a container (e.g. a rotating drum or a continuous flow mill) or any other deposition method wherein the marking composition attaches, adheres or bonds to the surface of the seeds. Furthermore, the marking composition may be applied to the seeds by seed-coating machines used in agricultural facilities for seed treatment, as part of seed production processes, where the seeds can be blended in common seed coating materials (used for applying antimicrobial, or fungicidal substances as well as fertilizers, coloring and other purposes).

Alternatively, a marking composition may be applied to the seeds prior to or during the germination phase when the seeds are soaked in water or are maintained under moist conditions by adding a marking composition to the water coming in contact with the seeds.

In some embodiments, the at least one XRF identifiable marker is water-insoluble. In such cases, a water-based composition comprising one or more water insoluble markers may be in the form of an emulsion (e.g., a suspension, a dispersion or a colloid) for use in forming a material coating on an external surface of the seeds.

The water insoluble markers may be additionally or alternatively be provided attached or bonded to an intermediate or bridging molecule that can bond or associate to an amino acid present in plant or plant tissues. Such bridging molecules may be selected amongst ionic surfactants, nonionic surfactants, Chitosan and Lignin.

As the seeds imbibe water during germination, the XRF identifiable marker (as well as the carrier) can be absorbed by the seed into the developing germ or seedling. The XRF identifiable marker then spreads and defuses to other parts of the growing germ (stalks, leaves and flowers) and may be detected by an XRF spectrometer.

In some embodiments, the concentration of the XRF identifiable marker in the water, in which the seed dipped or with which the seed is in contact, is between tens of ppm and tens of percent.

Thus, the invention further provides a seedling or a germ developed from a seed coated in accordance with the invention.

Also provided is a seedling or a germ having at least one tissue region thereof comprising an amount of at least one XRF identifiable marker, as defined herein. The seedling or germ, as known in the art, is a young plant developing out of a plant seed. The seedling may be differentiated from the seed (from which it develops) or from the adult plant (into which it develops) by having an embryonic root, an embryonic shoot and seed leaves.

Further provided is a seedling or a germ enriched with at least one XRF identifiable marker, as defined herein. The marker may be present in the seedling root, shoot and/or leaves.

As detailed herein, the invention provides an agricultural composition comprising the at least one XRF identifiable marker for marking or enriching a living plant with an XRF identifiable marker through irrigation or fertilization. A method for marking a living plant through irrigation involves the use of irrigation water comprising an amount of the XRF identifiable marker. Upon irrigation with the water, the marker becomes absorbed by the soil or growing medium and taken up by the roots of the living plant, spreading and diffusing to the tissues of the stem, stalks, leaves and flowers. The absorbed markers remain inside the plant tissues for an extended period of time depending, inter alia, on the concentration of marker in the irrigation water and the water uptake of the plant. As the markers present in the plant tissues cannot be washed off, detection by an XRF spectrometer becomes possible.

As the marker is present inside the tissues of the plant it may remain there during various stages of processing. For example, leaves and flowers may be harvested and dried in a first stage of processing (e.g. as in cannabis processing). As the flower or leaf is dried and loses water the concentration of the marker within the plant may increase allowing easier and more accurate identification of the marker.

Alternatively, a composition with one or more XRF identifiable markers may be incorporated into the growing medium or soil by, e.g., a plough or a rototiller, or may be placed on the soil surface as a topdressing or in a solid or granular form. In such implementations, uptake of the XRF identifiable marker by the roots will take place once the markers are dissolved by rain or irrigation water. The incorporation of the marker may be by adding a solid marker to the growing medium or soil (e.g., wherein the marker is mixed optionally with a solid carrier and thereafter mixed into the growing medium or soil—before the plant is planted or the seed allowed to germinate, or during the growth period of the plant), by adding at least one nutrient in the form of at least one XRF identifiable marker (e.g., wherein the at least one nutrient may comprise the marker or may be the marker), or by fertilizing the plant with at least one fertilizer composition comprising at least one XRF identifiable marker.

A composition of the invention may also be used for application, e.g., by spraying, onto the plant external surfaces (of plant parts such as stalks, stems, leaves, etc). The plant parts may be marked by applying a composition, as defined herein, to the external surface of the plant by spraying, brushing, dipping or by soaking the plant or plant parts prior to or after harvesting. For instance, the composition may be blended with liquid fertilizers, growth promoters, and/or pest- or herb-control materials that are sprayed on the plant.

The invention further provides a method of authenticating a seed or an explant having been marked with at least one XRF identifiable marker, the method comprising directing an X-ray signal to the explant and detecting and analyzing a (secondary) X-ray response signal from the explant, such that when the response signal corresponds to said at least one XRF identifiable marker, the explant is authenticated.

The XRF marking can be detected from an outer surface of the living plants or seed or explant or from inner layers of the living plant or seed or explant. Similarly, a suitable XRF reader can detect the markers from inside a package. For example, a package of marked seeds may be made of a laminated polymeric material wherein the markers are identified by a handheld XRF reader. The laminated polymeric material may be up to hundreds of microns thick and for heavier marker elements even thicker. Alternatively, the package or the wrap may be itself marked by an XRF marking wherein the markings of both the product in the package and the package itself can be measured by the same XRF reader, optionally by taking a single spectrum. Systems and techniques for marking polymeric materials suitable for marking packages are described in International Patent Applications PCT/IL2017/051112 or any US application derived therefrom and in U.S. provisional application 62/874,141 which are incorporated herein by reference.

In an aspect of the present invention the marking of seeds, explants and plants provides a tool for authentication as they change hands throughout the supply chain from the cultivator, through various distributors and processing facilities to, possibly, the end user. The plant or seed may be marked a single time at one location (e.g. the grower/cultivator), and may be detected at a second location/facility further along the supply chain (e.g. distributor or processing facility). Alternatively, the plant or seed may be marked in a plurality of locations wherein in each location the same or different markers are applied to the plant or seed. For example, the seed may be marked prior to germination, the germ may be marked a second time, the plant as it grows may be also marked one or more times, and then marked again after harvesting. In another example, the plant may also be marked during and after processing so that it may be read at various locations up to the end user.

The method of the present invention may be used for managing and supervising a chain of supply of plants or products made of or containing plant ingredients. The marking on the plant may be used to identify the seed, plant or plant product and their supplier. A system for managing a supply chain for plants may include a database system (central or distributed) where data relating to plants and their marking is stored. For example, the database system may record past and current locations of a plant, a package or a batch of plants and plant products as well as the future destinations (e.g. distributors and buyers). For that purpose, the device reading the marking (e.g. an XRF analyzer) may communicate with the database system. The database system may be an on-premises, cloud based system or a distributed ledger. In an example, the database system may be a distributed blockchain system wherein a plurality of parties store and access the relevant data. In such a blockchain system a plurality of parties (for example, parties which are members of the same supply chain) may store and access data wherein the data stored is immutable, easily verifiable and, due the distributed design, inherently resistant to modification. In an example, the marking and the blockchain system of the present invention may be used for managing a supply chain of cannabis plants and seeds and cannabis products. The parties to the blockchain system may include cultivators, laboratories and processing facilities, distributers, pharmaceutical companies pharmacies, traders, delivery companies, governmental agencies, and even end users. A cannabis plant or cannabis product may be recorded as it is changes hands between the parties. In an example the marking of the cannabis plant or product is read (detected) by a suitable XRF device and recorded every time it changes hands along the supply chain and recorded (e.g. automatically) on the blockchain allowing each party to easily verify the provenance and complete history of the plant or product. Blockchain systems that are suitable for managing a supply chain of marked objects and products are described in International Patent Applications PCT/IL2018/050499 and PCT/IL2019/050283 or any US applications derived therefrom, which are incorporated herein by reference.

In an example, methods of the present invention may be used to manage and supervise a supply chain of cannabis plants and cannabis products. The cannabis plant and seed may be marked at one or more locations. For example, the cannabis seeds and cannabis seeds packages may be marked at one location (using one method), and the plant may be marked a second time (by the same or different code) during germination and growth (e.g., by irrigation) at a different location. As the plant or plant part/ingredient progress along the supply chain (e.g. to various suppliers, distributors, processing facilities), the markings can be read, thereby authenticating the source of plant, its various distributors, its processing facilities and so on. Additionally, the marking may indicate the species and strain of the plant (different strains may be indistinguishable from each other particularly during the early stages of growth) and the concentration levels of the various cannabinoids in the plant. In an example, the marking may indicate whether the plant or product is intended for medical use and furthermore, the specific medical uses for which it is intended.

An XRF spectrometer or analyzer may be used to detect and measure markers present at a volume extending beyond the surface of a seed or an explant, wherein the depth of the volume beneath the surface is determined by the energy of the radiation emitted towards seed or explant and the response signal emitted by the markers, and the composition of the marker material and the plant as well as additional factors (e.g. the geometrical configuration of the detector and emitter in the XRF spectrometer and the measured sample). The depth of penetration of radiation incoming towards a sample increases for lighter materials (e.g. organic materials) and so does the distance that the response signal may travel inside the sample towards the detector in the XRF analyzer. Hence XRF-spectroscopy is particularly suitable for inspecting and detecting markers present inside a sample and particularly samples of organic material as it can detect and measure markers inside the bulk of the sample. A plurality of marker materials (elements) (e.g. elements heavier than Mg) may be detected within a bulk of organic material at a depth (distance from the external surface of the bulk) ranging from hundreds of microns to few millimeters or tens of millimeters, and in some cases even more. An XRF analyzer detecting marker elements may by a mobile Energy Dispersive X-Ray Fluorescence EDXRF device (for example a handheld EDXRF). The XRF analyzer may also be a benchtop device. An XRF analyzer suitable for detecting and measuring markers present on the surface or inside a plant (in various depths) is described in International Patent Application PCT/IL2017/051050 or any US applications derived therefrom, which are incorporated herein by reference. Additionally, the XRF analyzer may be a Wavelength Dispersive X-Ray Fluorescence (WDXRF) device.

Products derived from plants or plant parts that are enriched or marked with a marker, as disclosed herein, may be in a form of dry plant material, e.g., such as dry leaves, flowers, stems, stalks, shoots and other plant parts; in a form of an infusion, wherein marked leaves, flowers, stems, stalks, shots and other plant parts are infused in water; in a form of an extract, wherein active materials are extracted from the plant parts and the markers are extracted therewith; or in any other fresh form, wherein the marked plant is provided for use as is. The marking in any such product derived from a plant or an explant or a seed or a seedling marked according to the invention may be detected and identified by a suitable XRF reader.

The invention further provides a method for identifying a production and commercial history of a plant-based product, the method comprising
treating a seed or an explant with a formulation comprising a first XRF-identifiable marker at a first time point, under conditions permitting embedding said first marker in the seed surface or in the explant surface or tissue; wherein the first marker encoding at least one parameter relating to the seed or explant or a growing process relating thereto;
at a second time point, optionally treating a seedling grown from said seed with a second XRF-identifiable marker under conditions permitting embedding said second marker in a tissue of said seedling; wherein the second marker encoding at least one parameter relating to seedling growing stage; and
analyzing the presence of the first and second XRF-identifiable markers in a plant derived from said seed, explant or seedling or in a product manufactured therefrom.

Also provided is a method for managing a chain of supply of a plant or a product derived therefrom, the method comprising marking a seed or an explant of said plant, and/or marking a seedling developed from said seed or explant with at least one XRF-identifiable marker, wherein the marker is not naturally present in the seed, and wherein the marker is in an amount sufficient to enable XRF identification in a tissue derived from said living plant at a time after said marking, to thereby obtain at least one information relating to the plant or product chain of supply.

In some embodiments, the plant is cannabis.

In some embodiments, the product is a cannabis-based product, as defined herein.

Also provided is a cannabis seedling or a germ having at least one tissue region thereof comprising an amount of at least one XRF identifiable marker, wherein the marker is not naturally present in said seedling or germ.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-B show the detected marker signal intensity on the seed and the primary root (after washing with distilled water) as well as a spectrum from an unmarked seed.

FIG. 2 depicts graphs D₂-0, D₂-4, D₂-7 and D₂-19 showing the marker (Br) signal intensity vs. energy measured from typical leaves of plant 2 picked on day 0 (before marking), day 4, day 7, and day 19 of the 14 day period, respectively.

FIG. 3 depicts graphs D₃-0, D₃-3, D₃-9 and D₃-14 showing the marker (Br) signal intensity Vs. energy measured from typical leaves of plant 3 picked on day 0 (before marking), day 3, day 9, and day 14 of the 14 day period, respectively.

### DETAILED DESCRIPTION OF EMBODIMENTS

### General Marking Compositions

Compositions of the invention have been prepared utilizing a variety of XRF markers. Some of the markers were water-soluble and some were water-insoluble. The following are non-limiting examples of markers used according to the invention.

Soluble Markers:

Bis-bromo-ethylpropane-diol; Co(NO₃)₂*6H₂O; Ni(NO)₂*6H₂O; Y(NO₃)₂*6H₂O; SnCl₂*2H₂O; NH₄Br; NaCl; KI; Cs₂CO₃; Na₂O₄Se.

Non-Soluble Markers:

CoAcAc; NiAcAc; Tin Ethyl hexanoate; Tribromoanyline; Trichloroaniline; WO₂; Nb₂O₅

### Example 1—Seeds

Six types of seeds where marked: two different types of tomato seeds, two different types of paper seeds, and two different types of watermelon seeds. Each type of seeds was marked with a marking composition including 2 to 3 molecules, each molecule comprising a one or two marker elements. The following 7 marker molecules where used:

Bi Germanium Oxide—Bi₄Ge₃O₁₂; Gallium Oxide—Ga₂O₃; Strontium molybdate—SrMoO₄; Yttrium oxide—Y₂O₃: Niobium oxide—Nb₂O₅; Bismuth (III) citrate—BiC₆H₅O₇; Bismuth Oxide—Bi₂O₃.

Six marking compositions each comprising 2-3 of these molecules (defining 6 codes for the respective six seed types) were prepared. The marking compositions were dispersed/dissolved/suspended in an industrial coating composition for seeds (i.e. a coating composition used in the industry), wherein the final concentrations of each marker element in the blend were between 0.1% to 0.15% by weight. The coating composition with the marking compositions was applied to the seeds by a rotating drum. Each batch of seeds was inspected by a handheld XRF device directly (outside a package) and inside their package. The seed package comprised a laminated polymeric material including metalized barrier layer with a total thickness of 100 μm. The intensity of the signals received by the XRF device was reduced when measured inside the package was reduced by few percent to 50% (compared to the non-packaged samples), yet all six seed batches were identified both outside and inside the package.

In a similar fashion, other XRF markers were used for the purpose of marking seeds.

### Example 2—Seeds (Solution)

Melon and watermelon seeds were marked by soluble marking compositions dissolved in a water-based coating composition (one which is used in the industry). Each type of seeds was marked by a marking composition including one or two molecules, each molecule comprising one marker element. The following 3 molecules were used: Sodium Bromide—NaBr; Yittrium (III) nitrate hexahydrate—Y(NO₃)₃*6H₂O; and Cobalt (III) nitrate hexahydrate—Cₒ(NO₃)₂*6H₂O.

The seeds were coated by coating composition by a rotating drum or alternatively by an airbrush. The concentration of all markers in the coating composition was between 1000 ppm to 2000 ppm. Each batch of coated seeds was inspected by a handheld XRF device directly (outside a package) and inside their package. The seed package comprised a laminated polymeric material including metalized barrier layer with a total thickness of 100_{μm}. The intensity of the signals received by the XRF device was reduced when measured inside the package was reduced by few percent to 50% (compared to the non-packaged samples). All batches of marked seeds were identified both outside and inside the package.

### Example 3—Germination

Mung bean (Vigna radiata) seeds where marked prior and during germination by a marking composition comprising Chromium (III) chloride hexahydrate (CrCl₃*H₂O) in several concentrations. The seeds were inspected by a handheld XRF device prior to marking and no significant Cr signal marker was detected.

A first batch of seeds where soaked in water for 24 hours and then coated by a seed coating composition including the marking composition in concentration 5000 ppm. The seeds were placed in a sprouting vessel for germination. The seeds/germs were kept moist watering the sprouting vessel with distilled water. After two days the germ was inspected by a handheld XRF device and a strong Cr signal was detected measuring the seed. A weaker Cr signal was detected on the primary root coming out of the seed (the radicle).

A second batch of seeds was marked by soaking the seeds in water with the marking composition for 20 hour period and then watering the seeds (placed on a moist cotton wool substrate) by water containing the marking composition. This was done for three different concentrations of the marking composition in the water (for both soaking seed and watering the germ) 20 ppm and 100 ppm. The seeds and the primary roots coming out of seeds were inspected by handheld XRF device before and after washing with distilled water. The marker was detected in the seed, the primary root, of germ for all 3 concentrations both before and after washing. The signal received from the seeds and primary roots decreased after washing by few percent and up to 50%.

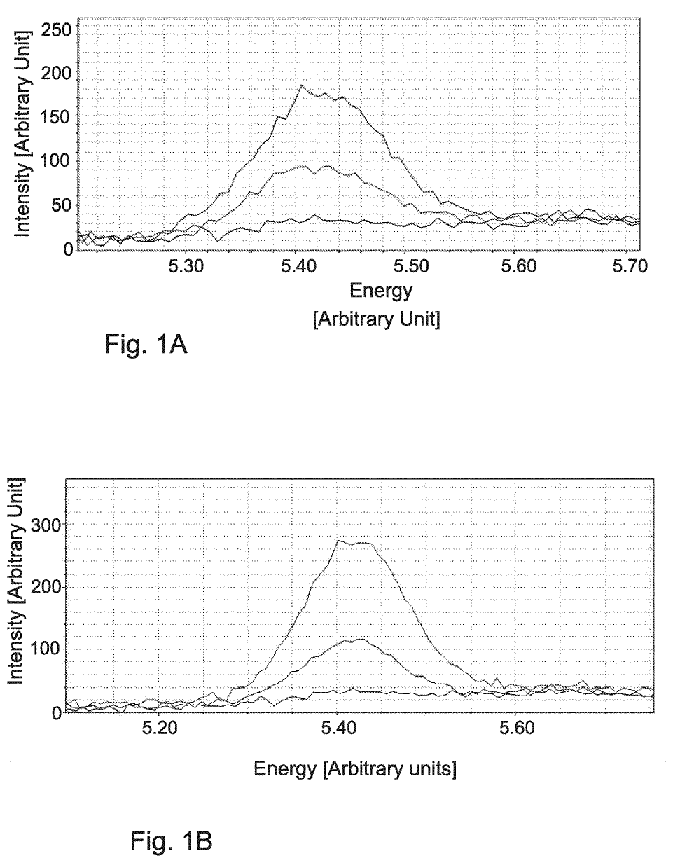

FIGS. 1A and 1B show the detected marker signal intensity on the seed and the primary root (after washing with distilled water) as well as the spectrum from an unmarked seed. FIG. 1A shows the marker signal intensity Vs. energy for the seed (top line), primary root (middle line) marked by a solution with marker concentration of 20 ppm and an unmarked seed bottom line. As shown in FIG. 1A the Cr signal intensity of a seed marked with 20 ppm solution was higher by 124% than signal received from an unmarked seed. The signal received from the primary root (after washing) was higher than the unmarked seed by 57%. FIG. 1B shows the marker signal intensity Vs. energy for the seed (top line), primary root (middle line) marked by 100 ppm solution and an unmarked seed (bottom line). The Cr signal intensity measured from the seed marked by a 100 ppm solution was higher by 218% than the signal intensity of an unmarked seed, while the Cr signal intensity measured from the primary root was higher by 60%.

A third batch of seeds was marked by soaking the seeds in water containing the marking composition for a period of 13 hours. The seeds were then placed on a moist cotton wool substrate and watered with distilled water (without a marking composition). This was done for three different concentration 20 ppm, 50 ppm, and 100 ppm. The germs were inspected by a handheld XRF device. The marker was detected on the seed.

### Example 4—Plants

Three spearmint (Mentha spicata) plant in a pot with potting mix was marked by irrigation (watering the potting mix). The marking composition dissolved in the irrigation water for all three plants included Calcium bromide (CaBr₂). The plants were inspected by a handheld XRF device daily or every few days. In each inspections three leaves from three different stalks were inspected measuring the Br signal in the acquired spectrum. The leaves were inspected both before and after washing with tap water without any processing. No significant difference was found between the washed and unwashed leaves. In all leaves that were inspected (for three plants) a very small signal of Br was detected prior to marking. This signal (for all three plants) was much smaller (up to orders of magnitude) than the signal of the marked plants.

Plant 1 was watered daily for two weeks with tap water with CaBr₂ in concentration of 5000 ppm. After two weeks the ratio between the Br signal intensity (averaged over 3 leaves) to the Br signal intensity prior to marking was 496. From that point onward the plant was watered with tap water (without a marker). The ratio between the Br signal intensity to the Br signal prior to marking decreased gradually to about 320 after 29 days regular irrigation (without a marker).

Three leaves were picked after five days of irrigation with marked water and then dried for two weeks in room conditions (temperature and lighting). These leaves were inspected before and after drying. The signal intensity of the marker increased significantly after drying. On average (over the three leaves) the ratio of the marker signal to the background increased by 34%.

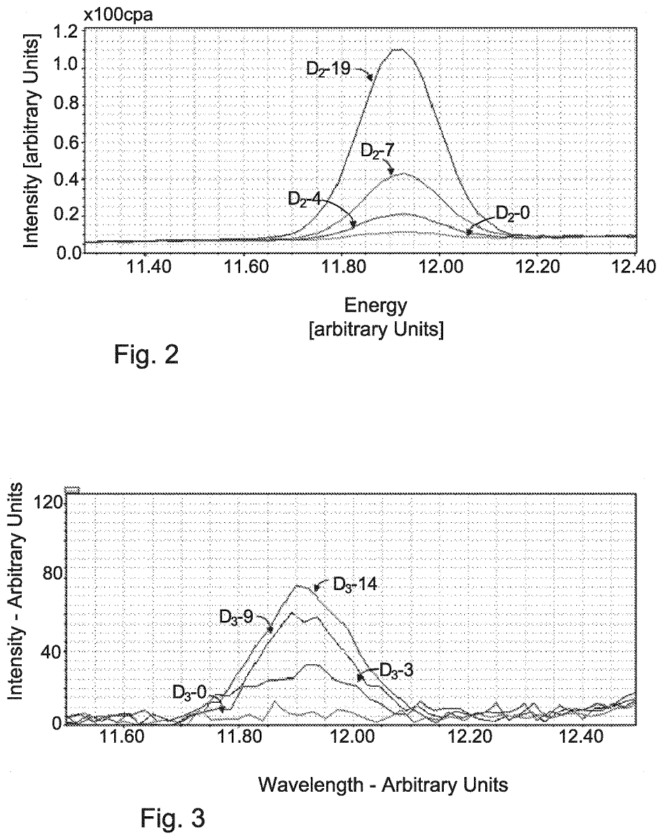

Plant 2 was watered daily over a 19 day period with tap water with 50 ppm of CaBr₂. Three leaves (from 3 different stalks) were inspected each day by a handheld XRF before each watering. The ratio of the Br signal intensity to the Br intensity increased to 16.3 (that is, by more that 1500%) on that period. After the 19 day period the plant was watered daily with regular tap water (without the marker) for further 10 days (29 days in total). No significant decrease in the marker signal intensity to was detected after the 10 day period. FIG. 2 depicts graphs D₂-0, D₂-4, D₂-7 and D₂-19 showing the marker (Br) signal intensity Vs. energy measured from typical leaves of plant 2 picked on day 0 (before marking), day 4, day 7, and day 19 of the 14 day period respectively.

Three leaves were picked on the after five days of irrigation with marked water and then dried for two weeks in room conditions (temperature and lighting). These leaves were inspected before and after drying. The ratio of the marker signal intensity to marker intensity prior to marking increased on average (over the three leaves) by 21%.

Plant 3 was watered daily over a 14 day period with tap water wherein 50 ppm of CaBr₂ marker were dissolved in the water every third day (in total 4 times). Three leaves (from 3 different stalks) were inspected by a handheld XRF before each watering. The average (over three leaves each day) marker signal intensity increased, relatively to the unmarked plant (as measured on day 0 before marking), by 569% on day 3, by 774% by day 9, and by 1052% by day 14. FIG. 3 depicts graphs D₃-0, D₃-3, D₃-9 and D₃-14 showing the marker (Br) signal intensity Vs. energy measured from typical leaves of plant 3 picked on day 0 (before marking), day 3, day 9, and day 14 of the 14 day period respectively.

1-42. (canceled)

43. A composition comprising at least one XRF identifiable marker for use in a method selected from:
a method of marking a seed or a seedling under conditions permitting development of a plant enriched with the XRF identifiable marker;
a method of enriching a living plant with the XRF identifiable marker through irrigation or fertilization; and
a method of applying a marker composition to a surface of a living plant, pre- or post-harvest.

44. The composition according to claim 43, adapted to be taken up by a living plant.

45. The composition according to claim 43, further comprising at least one additive selected from adhesive materials, coloring materials, pesticides, fertilizers, and nutrients.

46. The composition according to claim 43, for application onto a surface region of an explant capable of developing into a viable plant.

47. The composition according to claim 46, wherein the explant is selected from a shoot tip, an axillary bud, a somatic embryo and a seed.

48. The composition according to claim 43, for application onto a surface region of a living plant.

49. The composition according to claim 43, wherein the plant is cannabis.

50. A method for XRF marking an explant with at least one XRF identifiable marker, the method comprising forming a coating of the at least one XRF identifiable marker on at least a region of the explant, wherein the at least one XRF identifiable marker is optionally comprised within at least one carrier material.

51. The method according to claim 50, wherein the explant is a seed.

52. A seed coated on its surface with a coating comprising at least one XRF identifiable marker, wherein the marker is not naturally present in the seed.

53. The seed according to claim 52, wherein the coating comprises at least one carrier material selected amongst natural or synthetic polymers.

54. The seed according to claim 53, wherein the at least one carrier material comprises cellulose and cellulose-derived materials, chitosan, acacia gum, starch, polyethylene glycol, polyvinyl acetate and polyvinylpyrrolidone.

55. A seedling or a germ having at least one tissue region thereof comprising an amount of at least one XRF identifiable marker, wherein the marker is not naturally present in said seedling or germ.

56. A method for XRF marking a living plant with at least one XRF identifiable marker, the method comprising watering said living plant with waters enriched with at least one XRF identifiable marker to enable uptake of the marker by the living plant, wherein the marker is not naturally present in the seed, and wherein the marker is an amount sufficient to enable XRF identification in a tissue derived from said living plant.

57. The method according to claim 56, wherein watering is achieved by irrigation.

58. A method for XRF marking a living plant with at least one XRF identifiable marker, the method comprising incorporating at least one XRF identifiable marker in a growing medium or soil of said living plant and allowing uptake of the marker by the living plant, wherein the marker is not naturally present in the seed, and wherein the marker is in an amount sufficient to enable XRF identification in a tissue derived from said living plant.

59. A method for XRF marking a living plant with at least one XRF identifiable marker, the method comprising applying at least one XRF identifiable marker onto a surface region of said living plant, wherein the marker is not naturally present in the seed, and wherein the marker is in an amount sufficient to enable XRF identification in a tissue derived from said living plant.

60. A method of authenticating a seed or an explant having been marked with at least one XRF identifiable marker, the method comprising directing a X-ray signal to the explant and detecting and analyzing a (secondary) X-ray response signal from the explant, such that when the response signal corresponds to said at least one XRF identifiable marker, the explant is authenticated.

61. A method for identifying a production and commercial history of a plant-based product, the method comprising
treating a seed or an explant with a formulation comprising a first XRF-identifiable marker at a first time point, under conditions permitting embedding said first marker in the seed surface or in the explant surface or tissue; wherein the first marker encoding at least one parameter relating to the seed or explant or a growing process relating thereto;
at a second time point, optionally treating a seedling grown from said seed with a second XRF-identifiable marker under conditions permitting embedding said second marker in a tissue of said seedling; wherein the second marker encoding at least one parameter relating to seedling growing stage; and
analyzing the presence of the first and second XRF-identifiable markers in a plant derived from said seed, explant or seedling or in a product manufactured therefrom.

62. A method for managing a chain of supply of a plant or a product derived therefrom, the method comprising marking a seed or an explant of said plant, and/or marking a seedling developed from said seed or explant with at least one XRF-identifiable marker, wherein the marker is not naturally present in the seed, and wherein the marker is in an amount sufficient to enable XRF identification in a tissue derived from said living plant at a time after said marking, to thereby obtain at least one information relating to the plant or product chain of supply.
